(19) United States
(12) Patent Application Publication
(10) Pub. No.: US 2026/0224400 A1
(43) Pub. Date: Aug. 6, 2026

(54) OCULAR CANNULA GUIDE, APPLICATOR, AND MARKING INSTRUMENT

(71) Applicant: Genentech, Inc., South San Francisco, CA (US)

(72) Inventors: Thomas E. Meyer, Philadelphia, PA (US); Mark Hedgeland, King of Prussia, PA (US); Justin A. Creel, King of Prussia, PA (US); John Dale Miser, Sellersburg, IN (US); Peter James Meinz, Minneapolis, MN (US); Olivia Ahrens, Cincinnati, OH (US); Jerome Joseph Schafer, Cincinnati, OH (US); Kyle Stewart, Cincinnati, OH (US); Thomas Anthony Gernetzke, Cincinnati, OH (US)

(21) Appl. No.: 19/631,738

(22) Filed: Mar. 27, 2026

Related U.S. Application Data
(63) Continuation of application No. PCT/US24/48755, filed on Sep. 27, 2024.
(60) Provisional application No. 63/541,403, filed on Sep. 29, 2023.

(51) Int. Cl. A61F 9/007

(52) U.S. Cl. CPC A61F 9/00736

(57) ABSTRACT

An apparatus includes a pair of rigid legs extending parallel with each other along a plane. Each leg of the pair of legs has a sharp tip. The apparatus also includes head, including a top surface, a bottom surface, a pair of end surfaces, a pair of side surfaces, and a guide notch extending upwardly from the bottom surface. The guide notch is configured to cooperate with a surface of a sclera of a patient's eye to define a guide opening that is oriented transversely relative to the plane associated with the legs and that is sized to receive a cannula having a generally flat profile. The head further includes a pair of receptacles extending laterally inwardly from respective side surfaces of the pair of side surfaces. Each receptacle of the pair of receptacles is configured to engage a deployment instrument.

### BACKGROUND

The human eye comprises several layers. The white outer layer is the sclera, which surrounds the choroid layer. The retina is interior to the choroid layer. The sclera contains collagen and elastic fiber, providing protection to the choroid and retina. The choroid layer includes vasculature providing oxygen and nourishment to the retina. The retina comprises light sensitive tissue, including rods and cones. The macula is located at the center of the retina at the back of the eye, generally centered on an axis passing through the centers of the lens and cornea of the eye (i.e., the optic axis). The macula provides central vision, particularly through cone cells.

Macular degeneration is a medical condition that affects the macula, such that people suffering from macular degeneration may experience lost or degraded central vision while retaining some degree of peripheral vision. Macular degeneration may be caused by various factors such as age (also known as “AMD”) and genetics. Macular degeneration may occur in a “dry” (nonexudative) form, where cellular debris known as drusen accumulates between the retina and the choroid, resulting in an area of geographic atrophy. Macular degeneration may also occur in a “wet” (exudative) form, where blood vessels grow up from the choroid behind the retina. Even though people having macular degeneration may retain some degree of peripheral vision, the loss of central vision may have a significant negative impact on the quality of life. Moreover, the quality of the remaining peripheral vision may be degraded and, in some cases, may disappear as well. It may therefore be desirable to provide treatment for macular degeneration to prevent or reverse the loss of vision caused by macular degeneration. In some cases, it may be desirable to provide such treatment in a highly localized fashion, such as by delivering a therapeutic substance in the subretinal layer (under the neurosensory layer of the retina and above the retinal pigment epithelium) directly adjacent to the area of geographic atrophy, near the macula. However, since the macula is at the back of the eye and underneath the delicate layer of the retina, it may be difficult to access the macula in a practical fashion.

While a variety of surgical methods and instruments have been made and used to treat an eye, it is believed that no one prior to the inventors has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a perspective view of an example of an instrument for subretinal administration of a therapeutic agent from a suprachoroidal approach;

FIG. 2 depicts a perspective view of the instrument of FIG. 1, mounted near a patient, with a combination of medical equipment;

FIG. 3 depicts a perspective view of an example of a guide tack that may be used to guide the cannula of the instrument of FIG. 1 during performance of a procedure for subretinal administration of a therapeutic agent;

FIG. 4 depicts a front elevational view of the guide tack of FIG. 3;

FIG. 5 depicts a bottom plan view of the guide tack of FIG. 3;

FIG. 6 depicts a side elevational view of the guide tack of FIG. 3;

FIG. 7 depicts a cross-sectional view of the guide tack of FIG. 3, taken along line 7-7 of FIG. 6;

FIG. 8 depicts a perspective view of an example of a deployment instrument that may be used to deploy the guide tack of FIG. 3 in the eye of the patient;

FIG. 9A depicts a top plan view of the deployment instrument of FIG. 8, showing a pair of lever arms of the deployment instrument in a closed state to grasp the guide tack of FIG. 3;

FIG. 9B depicts a top plan view of the deployment instrument of FIG. 8, showing the pair of lever arms in an open state to release the guide tack of FIG. 3;

FIG. 10 depicts a perspective view of an example of a marking instrument that may be used to mark the eye of the patient;

FIG. 11 depicts a side elevational view of a distal end of the marking instrument of FIG. 10;

FIG. 12 depicts a cross-sectional view of the distal end of the marking instrument of FIG. 10, taken along line 12-12 of FIG. 11;

FIG. 13 depicts a front elevational view of the distal end of the marking instrument of FIG. 10;

FIG. 14A depicts a top plan view of an eye of a patient, with the marking instrument of FIG. 10 being used to mark a guide tack deployment site and a sclerotomy site on the eye;

FIG. 14B depicts a top plan view of the eye of FIG. 14A, with the deployment instrument of FIG. 8 being used to deploy the guide tack of FIG. 3 in the eye;

FIG. 14C depicts a top plan view of the eye of FIG. 14A, with the guide tack of FIG. 3 deployed in the eye;

FIG. 14D depicts a top plan view of the eye of FIG. 14A, with a sclerotomy being provided at the sclerotomy site of FIG. 14A;

FIG. 14E depicts a top plan view of the eye of FIG. 14A, with the cannula of the instrument of FIG. 1 being guided into the sclerotomy of FIG. 14D via the guide tack of FIG. 3;

FIG. 15A depicts a cross-sectional side view of an eye of a patient;

FIG. 15B depicts a cross-sectional side view of the eye of FIG. 15A, with the guide tack of FIG. 3 deployed in the eye, and with a sclerotomy being performed;

FIG. 15C depicts a cross-sectional side view of the eye of FIG. 15A, with a cannula of the instrument of FIG. 1 being inserted through the sclerotomy opening and in between the sclera and choroid of the eye;

FIG. 15D depicts a cross-sectional side view of the eye of FIG. 15A, with the distal end of the cannula being positioned adjacent to a target location;

FIG. 15E depicts a cross-sectional side view of the eye of FIG. 15A, with a needle of the instrument of FIG. 1 being advanced through the choroid to access the subretinal space at the target location;

FIG. 15F depicts a cross-sectional side view of the eye of FIG. 15A, with the needle of FIG. 15E dispensing a first volume of leading bleb fluid to provide separation between a region of the retina and the choroid at the target location; and

FIG. 15G depicts a cross-sectional side view of the eye of FIG. 15A, with the needle of FIG. 15E dispensing a therapeutic agent between a region of the retina and the choroid at the target location.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description serve to explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

For clarity of disclosure, the terms “proximal” and “distal” are defined herein relative to a surgeon or other operator grasping a surgical instrument having a distal surgical end effector. The term “proximal” refers the position of an element closer to the surgeon or other operator and the term “distal” refers to the position of an element closer to the surgical end effector of the surgical instrument and further away from the surgeon or other operator.

Furthermore, the terms “about,” “approximately,” and the like as used herein in connection with any numerical values or ranges of values are intended to encompass the exact value(s) referenced as well as a suitable tolerance that enables the referenced feature or combination of features to function for the intended purpose described herein.

### I. Example of Instrument for Subretinal Administration of Therapeutic Agent

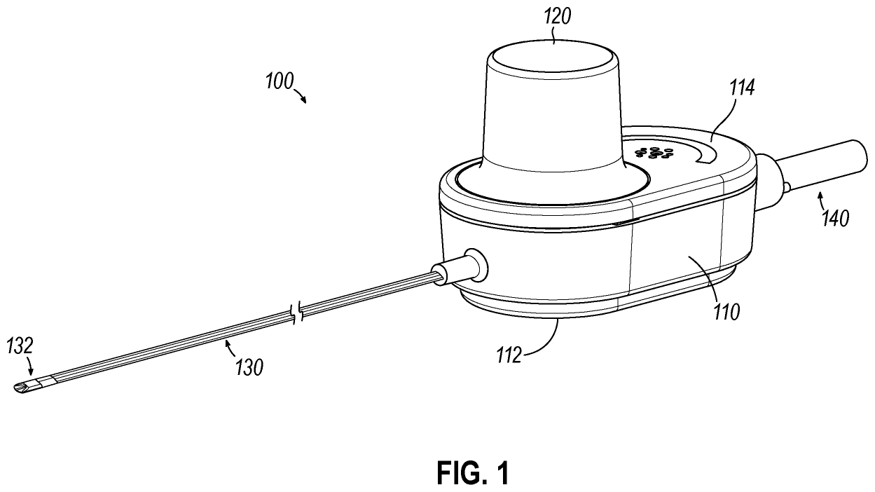

FIG. 1 shows an example of an instrument (100) that is configured for use in a procedure for the subretinal administration of a therapeutic agent to an eye (101) of a patient from a suprachoroidal approach. Instrument (100) comprises a body (110) and a flexible cannula (130) extending distally from body (110). Cannula (130) of the present example has a cross-sectional profile that is configured to enable cannula (130) to be passed atraumatically along the suprachoroidal space, as will be described in greater detail below. Cannula (130) is generally configured to support a needle (150) that is slidable within cannula (130), as will be described in greater detail below.

In the present example, cannula (130) comprises a flexible material such as Polyether block amide (PEBA), though any other suitable material or combination of materials may be used. Cannula (130) of the present example is flexible enough to conform to specific structures and contours of the patient's eye, yet cannula (130) has sufficient column strength to permit advancement of cannula (130) between the sclera (104) and choroid (106) of the patient's eye (101) without buckling. Cannula (130) includes a transversely oriented opening (not shown) near the distal end (132) of cannula (130). The opening may be formed by a U-shaped lateral recess in cannula (130), which leads to an open distal end (not shown) of a needle guide lumen within cannula (130). Distal end (132) is atraumatic such that distal end (132) is configured to provide separation between the sclera and choroid layers (104, 106) via blunt dissection, as will be described in greater detail below, to thereby enable cannula (130) to be advanced between such layers (104, 106) while not inflicting trauma to the sclera or choroid layers (104, 106).

By way of example only, cannula (130) may be configured and operable in accordance with at least some of the teachings of U.S. Pat. No. 10,226,379, entitled “Method and Apparatus for Subretinal Administration of Therapeutic Agent,” issued Mar. 12, 2019, the disclosure of which is incorporated by reference herein, in its entirety; U.S. Pat. No. 10,646,374, entitled “Apparatus and Method to From Entry Bleb for Subretinal Delivery of Therapeutic Agent,” issued May 12, 2020, the disclosure of which is incorporated by reference herein, in its entirety; U.S. Provisional Pat. App. No. 63/455,131, entitled “Apparatus for Subretinal Administration of Therapeutic Agent via Dual-Curved Needle,” filed Mar. 28, 2023, the disclosure of which is incorporated by reference herein, in its entirety; and/or in any other suitable fashion.

Needle (150) may be advanced distally to protrude from the opening of cannula (130). Needle (150) of the present example has a sharp distal tip (not shown) and defines a lumen (not shown). The distal tip may have a lancet configuration. In some other versions, the distal tip has a tri-bevel configuration or any other configuration as described in U.S. Pat. No. 10,226,379, the disclosure of which is incorporated by reference herein, in its entirety. Still other suitable forms that the distal tip may take will be apparent to those skilled in the art in view of the teachings herein. In the present example, the generally rectangular, generally elliptical, or otherwise generally flat cross-sectional profile of cannula (130) prevents cannula (130) from rotating about the longitudinal axis of cannula (130) when cannula (130) is disposed in the suprachoroidal space as will be described in greater detail below. This provides a consistent and predictable orientation of the opening of cannula (130), thereby providing a consistent and predictable exit path for needle (150) when needle (150) is advanced distally relative to cannula (130) as will be described in greater detail below. By way of further example only, needle (150) may include one or more preformed bends in accordance with at least some of the teachings of U.S. Pat. No. 10,478,553, entitled “Apparatus for Subretinal Administration of Therapeutic Agent via a Curved Needle,” issued Nov. 19, 2019, the disclosure of which is incorporated by reference herein, in its entirety; and/or U.S. Provisional Pat. App. No. 63/455,131, entitled “Apparatus for Subretinal Administration of Therapeutic Agent via Dual-Curved Needle,” filed Mar. 28, 2023, the disclosure of which is incorporated by reference herein, in its entirety.

As shown in FIG. 1, instrument (100) of the present example further comprises an actuation knob (120) located at a top portion (114) of body (110). Actuation knob (120) is rotatable relative to body (110) to thereby selectively translate needle (150) longitudinally relative to cannula (130). In particular, actuation knob (120) is rotatable in a first angular direction to drive needle (150) distally relative to cannula (130); and in a second angular direction to drive needle (150) proximally relative to cannula (130). By way of example only, instrument (100) may provide such functionality through knob (120) in accordance with at least some of the teachings of U.S. Pat. No. 10,646,374, the disclosure of which is incorporated by reference herein, in its entirety. Other suitable ways in which rotary motion of knob (120) may be converted to linear translation of needle (150) will be apparent to those skilled in the art in view of the teachings herein. Similarly, other suitable ways in which needle (150) may be actuated (150) longitudinally relative to cannula (130) will be apparent to those skilled in the art in view of the teachings herein. As also shown in FIG. 1, a conduit assembly (140) extends proximally from body (110). Conduit assembly (140) is configured to contain one or more fluid conduits (not shown) that are in fluid communication with needle (150). In some versions such fluid conduits are coupled with sources of leading bleb fluid and therapeutic agent.

### II. Example of System for Subretinal Administration of Therapeutic Agent

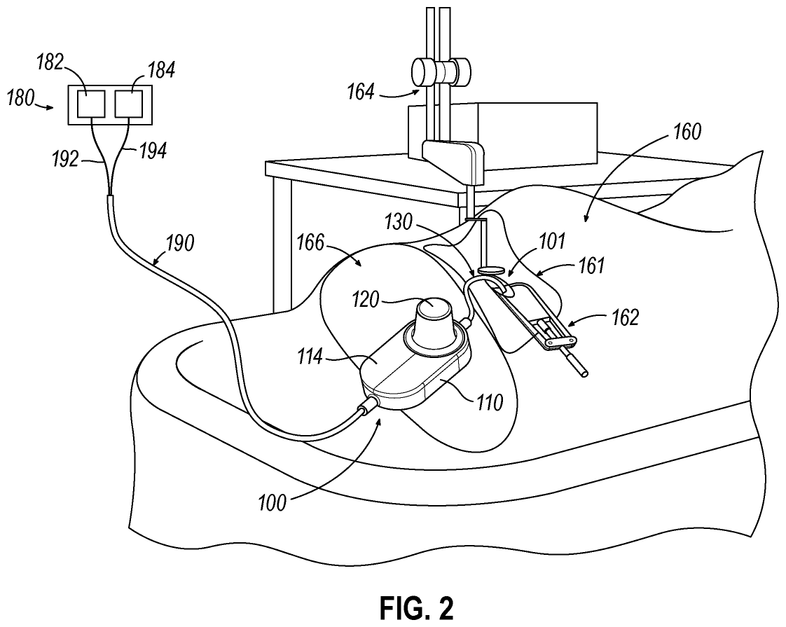

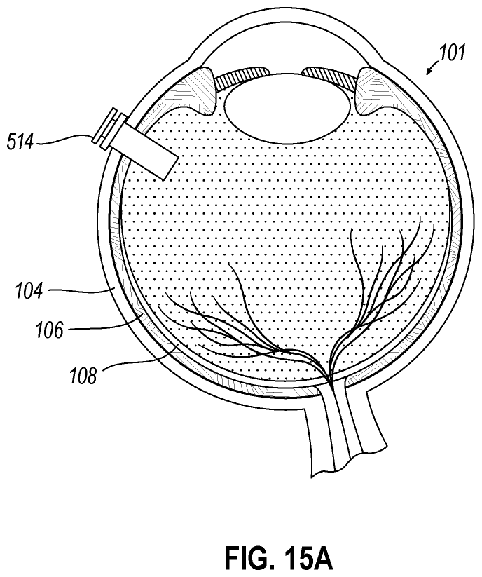

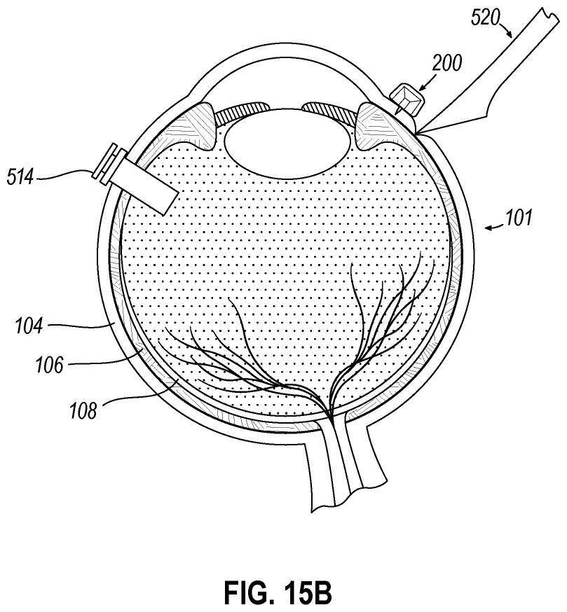

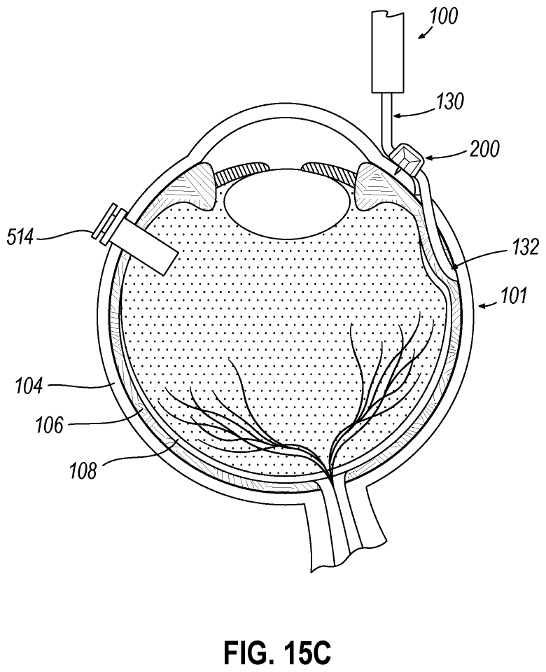

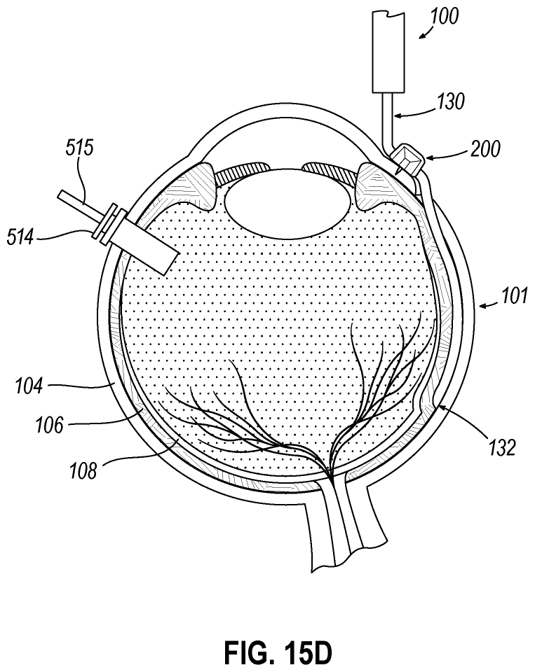

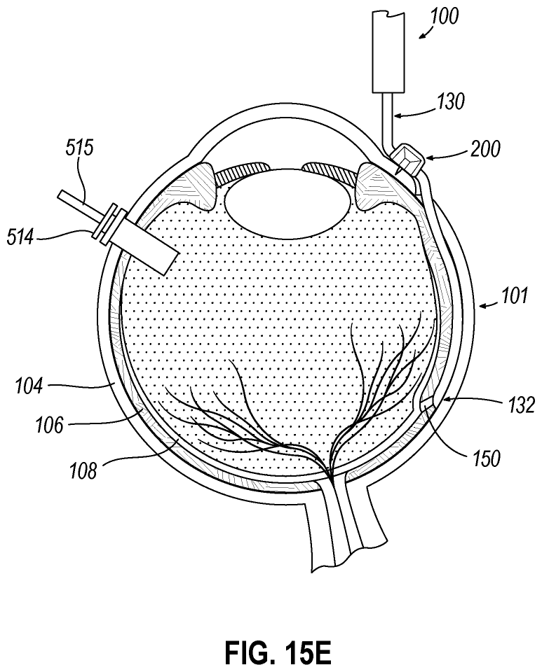

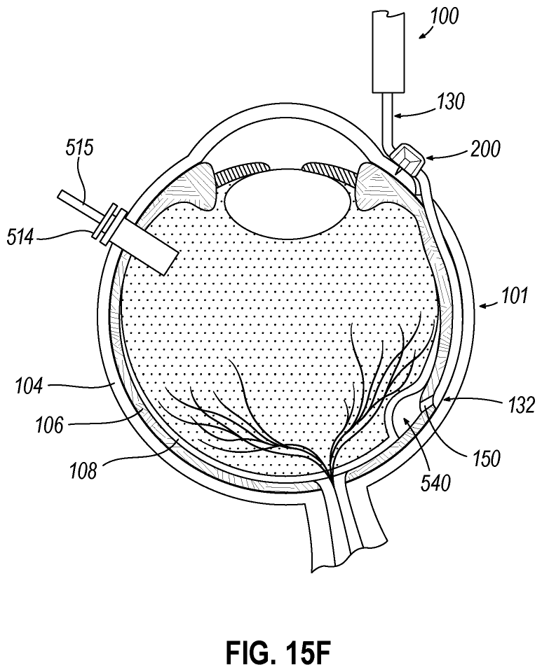

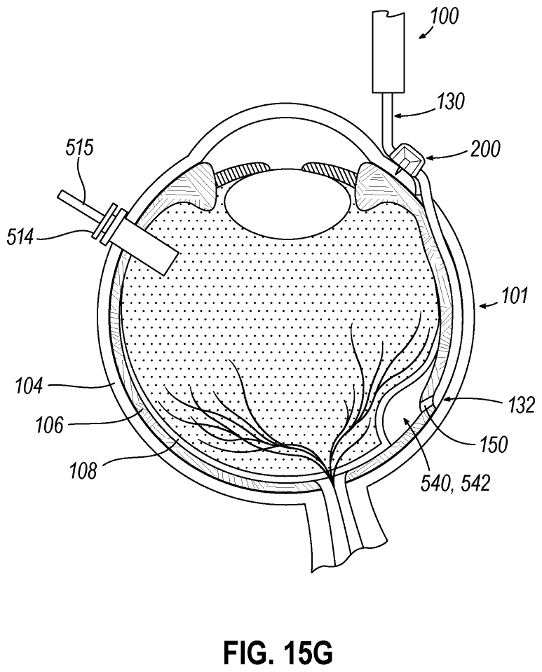

FIG. 2 shows a scenario where instrument (100) is positioned in relation to a patient. In this example, a drape (160) is disposed over the patient, with an opening (161) formed in drape (160) near the patient's eye (101). A speculum (162) is used to keep the eye (101) open. A fixture (164) is positioned adjacent to the eye (101). Fixture (164) may be used to secure instrumentation, such as a viewing scope, relative to the patient. A magnetic pad (166) is adhered to drape (160) near the opening (161) adjacent to the eye (101). Instrument (100) is placed on magnetic pad (166) and is removably secured thereto via magnetic attraction. In the present example, one or more permanent magnets (not shown) are positioned within body (110) near bottom potion (112); and these magnets are magnetically attracted to one or more ferrous elements (not shown) contained within magnetic pad (166). By way of example only, these magnets and magnetic pad (166) may be configured in accordance with at least some of the teachings of U.S. Pat. No. 10,806,629, entitled “Injection Device for Subretinal Delivery of Therapeutic Agent,” issued Oct. 20, 2020, the disclosure of which is incorporated by reference herein, in its entirety. Instrument (100) is oriented to enable insertion of flexible cannula (130) of instrument (100) into the eye (101). An example of a process for inserting and positioning cannula (130) in the eye (101) is described in greater detail below with reference to FIGS. 15A-15G.

In the present example, instrument (100) is coupled with a fluid delivery system (180) via conduit assembly (190). In this example, fluid delivery system (180) comprises a bleb fluid source (182) and a therapeutic agent fluid source (184). Bleb fluid source (182) is coupled with a bleb fluid conduit (192) of conduit assembly (190); and therapeutic agent fluid source (184) is coupled with a therapeutic agent conduit (194) of conduit assembly (190). Conduits (192, 194) are in fluid communication with needle (150). In some versions, fluid sources (182, 184) comprise syringes. In some other versions, fluid sources (182, 184) comprise separate reservoirs and one or more associated pumps and/or valves, etc.

### III. Example of Cannula Guide

In some instances, a suture loop assembly (not shown) may be installed in the eye (101) of a patient in order to stabilize and guide cannula (130) during insertion of cannula (130) into an incision (107) in the eye (101). Those skilled in the art will recognize that the formation of a suture loop assembly may be somewhat time consuming. In addition, it may be difficult to provide consistent spacing between the suture loops of a suture loop assembly; and between the suture loops and the eye (101). Such variations in spacing may yield variations in the entry angle and/or variations in the force required to insert cannula (130) through a suture loop assembly. It may therefore be desirable to provide a device that provides the stabilizing and guiding functionality of a suture loop assembly; yet that is faster and easier to install in the eye (101) than a suture loop assembly, yielding more consistent results. An illustrative example of such a device is described in greater detail below. The device described below may provide faster installation times, minimize variability due to surgeon technique, and require a lower level of expertise such that less surgeon training will be necessary. At the end of the procedure, the device may be removed from the eye (101) such that no foreign body will be left in the eye (101).

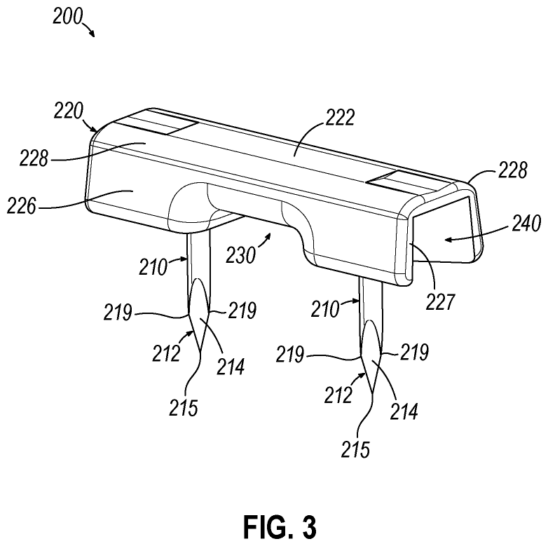

FIGS. 3-7 show an example of a guide tack (200) that may be used to guide cannula (130) through an incision (107) in the eye (101), instead of using a suture loop assembly. Guide tack (200) of this example includes a pair of legs (210) and a head (220), with legs (210) extending downwardly from head (220). The free end of each leg (210) includes a sharp tip (212). In the example shown, each sharp tip (212) has a tri-bevel configuration. In particular, each sharp tip (212) includes three facets (214) that converge together at a distal point (215), while each pair of adjacent facets (214) also converge at a corresponding proximal point (219). This tri-bevel configuration promotes relatively easy piercing of the eye (101) as the operator urges sharp tips (212) into the eye (101). Alternatively, sharp tips (212) may have any other suitable configuration that enables sharp tips (212) to pierce the tissue layers of the eye (101), including but not limited to various eye-piercing tip configurations known in the art. In some versions, sharp tips (212) and/or other portions of legs (210) may be textured to provide improved gripping of the tissue layers of the eye (101), such as the sclera (104).

Head (220) of the present example includes a top surface (222), a bottom surface (224), a pair of end surfaces (226), a pair of side surfaces (227), and a pair of chamfers (228) extending between top surface (222) and respective end surfaces (226). Head (220) further defines a guide notch (230) extending upwardly from bottom surface (224) of head (220). Guide notch (230) is oriented along an axis that is transverse to the plane defined between legs (210). When guide tack (200) is secured to the eye (101), guide notch (230) is configured to cooperate with an exposed surface (105) of the sclera (104) to define a guide opening. The guide opening defined by the surface (105) of the sclera (104) and guide notch (230) is sized and configured to slidably receive cannula (130).

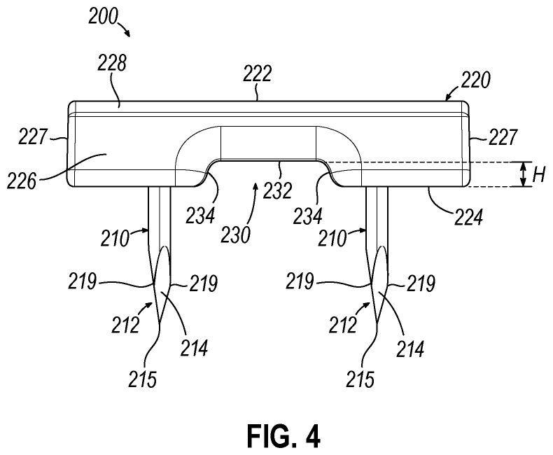

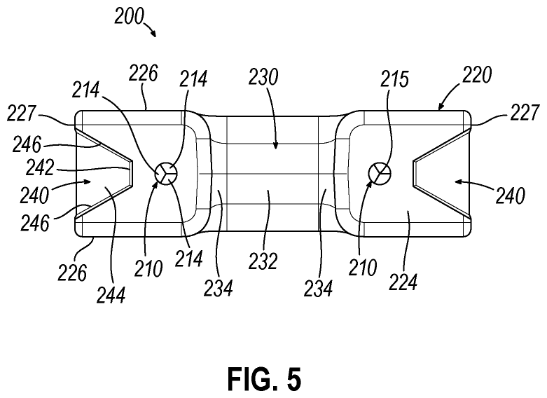

As best seen in FIGS. 4 and 5, guide notch (230) of the present example is defined by an upper notch surface (232) and a pair of outer notch surfaces (234). When viewed along the direction shown in FIG. 4, surfaces (232, 234) are configured such that guide notch (230) deflects outwardly from the centerline of guide tack (200), at the mid-region of guide notch (230) along the height of guide notch (230), along the plane defined between legs (210). This configuration may promote capture of cannula (130) within the guide opening defined by the surface (105) of the sclera (104) and guide notch (230), ensuring that cannula (130) remains at an appropriate distance from the surface (105) of the sclera (104).

When viewed along the direction shown in FIG. 5, surfaces (232, 234) are configured such that guide notch (230) deflects inwardly toward the centerline of guide tack (200), at the mid-region of guide notch (230) along the height of guide notch (230), along a plane that is perpendicular to the plane defined between legs (210). This configuration may minimize the amount of contact between cannula (130) and head (220), which may in turn minimize the amount of friction between cannula (130) and head (220) as cannula (130) is slid through the guide opening defined by the surface (105) of the sclera (104) and guide notch (230). Reducing friction may reduce the risk of cannula (130) inadvertently pulling guide tack (200) from the eye (101) as cannula (130) is slid through the guide opening defined by the surface (105) of the sclera (104) and guide notch (230). Despite this minimization of contact between cannula (130) and head (220), surfaces (232, 234) may still provide sufficient contact to maintain stability of cannula (130) when cannula (130) is disposed in the guide opening defined by the surface (105) of the sclera (104) and guide notch (230).

In the example shown, at the mid-region of guide notch (230), upper notch surface (232) is spaced apart from bottom surface (224) of head (220) by a vertical distance to define a minimum height (H) of the guide opening defined by the surface (105) of the sclera (104) and guide notch (230). In some versions, the minimum height (H) of the guide opening defined by the surface (105) of the sclera (104) and guide notch (230) may be substantially short. In this regard, the minimum height (H) of the guide opening defined by the surface (105) of the sclera (104) and guide notch (230) may be between about 0.27 mm and about 0.43 mm. For example, the minimum height (H) of the guide opening defined by the surface (105) of the sclera (104) and guide notch (230) may be about 0.35 mm. Such a relatively short minimum height (H) of the guide opening defined by the surface (105) of the sclera (104) and guide notch (230) may allow upper notch surface (232) to urge cannula (130) toward the sclera (104) and thereby improve the ability of guide tack (200) to maintain cannula (130) in a generally tangential orientation relative to a sclerotomy (107).

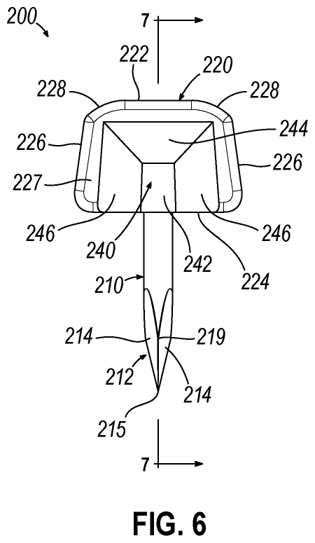

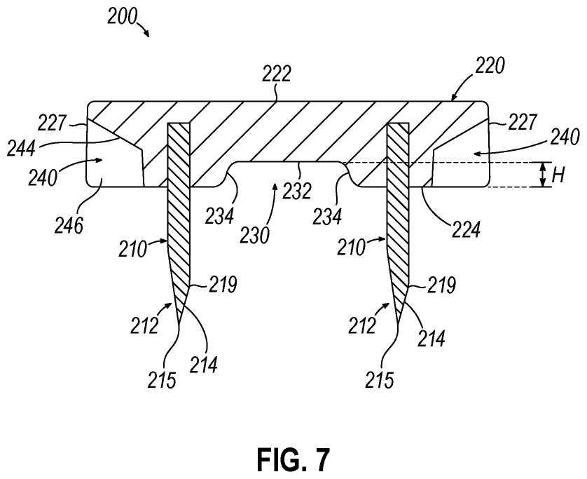

As best seen in FIGS. 5-7, head (220) of the present example further includes a pair of grasping features in the form of receptacles (240) extending laterally inwardly from respective side surfaces (227) of head (220), such that each side surface (227) is generally U-shaped. In the example shown, each receptacle (240) is defined by a corresponding laterally inner surface (242), a corresponding upper surface (244), and a corresponding pair of end surfaces (246), with each receptacle (240) being open along the bottom thereof. As shown in FIGS. 5-6, each pair of end surfaces (246) may be tapered toward each other in the laterally inward direction; and as shown in FIGS. 6-7, each upper surface (244) may be tapered downwardly in the laterally inward direction, while each laterally inner surface (242) may be substantially vertical (e.g., near-vertical). Each receptacle (240) may be configured to facilitate gripping of head (220) by the operator via a deployment instrument, such as deployment instrument (300) described below. For example, each receptacle (240) may be configured to selectively receive a corresponding portion of such a deployment instrument to enable the operator to grasp and manipulate guide tack (200) using the deployment instrument. In this manner, receptacles (240) may be used to assist with installing guide tack (200) on the eye (101).

By way of further example, guide tack (200) may be configured and operable in accordance with at least some of the teachings of U.S. Pat. No. 11,000,410, entitled “Guide Apparatus for Tangential Entry into Suprachoroidal Space,” issued May 11, 2021, the disclosure of which is incorporated by reference herein, in its entirety.

### IV. Example of Deployment Instrument

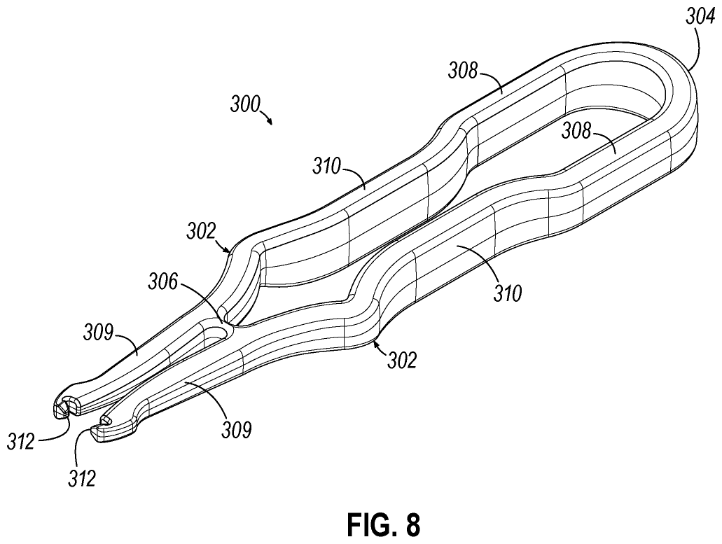

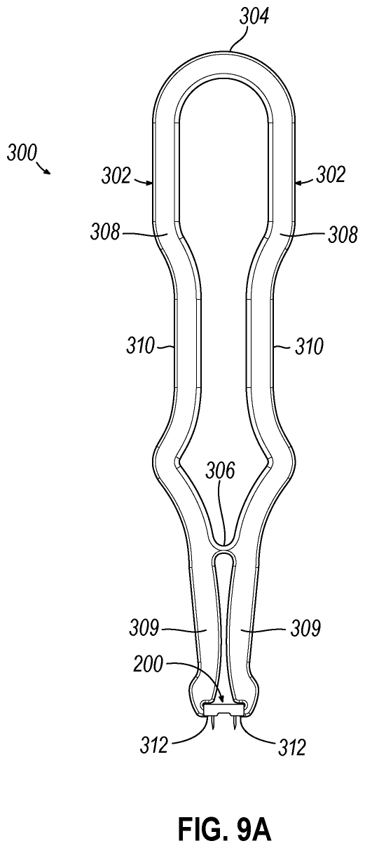

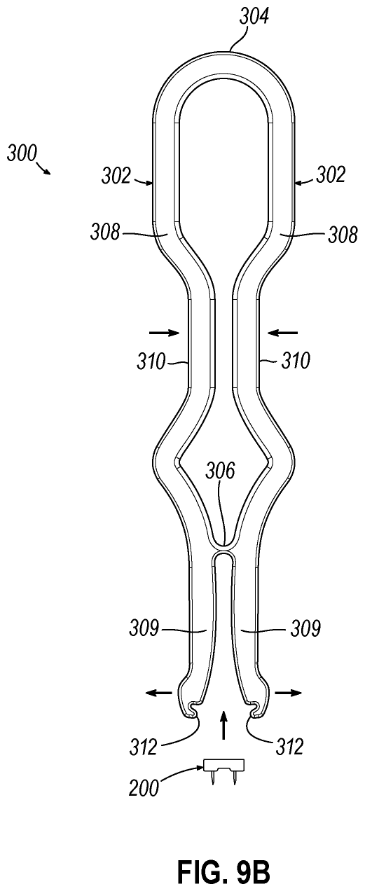

FIGS. 8-9B show an example of a deployment instrument (300) that may be used to install guide tack (200) in the eye (101). Instrument (300) of this example includes a pair of lever arms (302) extending distally from a proximal end (304). In the example shown, lever arms (302) are also pivotably coupled to each other at a discrete location along their respective lengths via a hinge in the form of living hinge (306), such that each lever arm (302) includes a respective proximal portion (308) that is proximal of living hinge (306) and a respective distal portion (309) that is distal of living hinge (306). In this manner, lever arms (302) may be pivotable relative to each other about living hinge (306) between a closed state (FIG. 9A) and at least one open state (FIG. 9B) in which proximal portions (308) are approximated toward each other and distal portions (309) are deflected farther away from each other. In some versions, living hinge (306) may be configured to resiliently bias lever arms (302) toward the closed state. While lever arms (302), proximal end (304), and hinge (306) are integrally formed together as a unitary (e.g., monolithic) piece in the example shown, it will be appreciated that lever arms (302), proximal end (304), and hinge (306) may be coupled to each other in any other suitable manner.

Proximal portions (308) of lever arms (302) include respective bowed regions defining respective finger rests (310) that extend inwardly toward each other to allow easy grasping and manipulation of proximal portions (308) of lever arms (302) by an operator's hand. For example, one finger rest (310) may receive a thumb of the operator's hand, while the other finger rest (310) may receive an index finger of the operator's hand, so that the operator may grasp both proximal portions (308) with a single hand and/or urge proximal portions (308) toward each other by performing a pinching motion with the thumb and index finger of the operator's hand. As discussed in greater detail below, due to the pivotable coupling of lever arms (302) to each other via living hinge (306), such urging of proximal portions (308) toward each other may cause distal portions (309) to pivot away from each other about living hinge (306).

Distal portions (309) of lever arms (302) include respective distal fingers (312) extending laterally inwardly toward each other. In this regard, each distal finger (312) may extend substantially perpendicularly to a longitudinal axis of deployment instrument (300). Distal fingers (312) are configured to cooperate with each other to selectively grasp guide tack (200). For example, each distal finger (312) may be configured to be selectively at least partially received within a corresponding receptacle (240) of guide tack (200) such that distal fingers (312) may thereby collectively grasp guide tack (200), at least when lever arms (302) are in the closed state. In some versions, each distal finger (312) may have a shape complementary to that of the corresponding receptacle (240), such that each distal finger (312) may be configured to mate with the corresponding receptacle (240) when received therein.

As shown, distal fingers (312) are spaced apart from each other even when lever arms (302) are in the closed state. In this regard, when lever arms (302) are in the closed state, distal fingers (312) may be spaced apart from each other by a gap sufficient to accommodate the portion of head (220) of guide tack (200) that is between receptacles (240). When lever arms (302) are in the closed state, finger rests (310) may be spaced apart from each other by a gap sufficient to allow approximation of proximal portions (308) toward each other and thereby increase the gap between distal fingers (312) enough to retract distal fingers (312) out of the corresponding receptacles (240) of guide tack (200).

In an example of a method of using instrument (300) to install guide tack (200) on the eye (101), lever arms (302) may initially be in the closed state. The operator may grasp proximal portions (308) by positioning the operator's thumb and index finger along the finger rests (310) of the proximal portions (308) of respective lever arms (302), and may perform a pinching motion with the operator's thumb and index finger to transition lever arms (302) to the open state to thereby increase the gap between distal fingers (312). The operator may then position each distal finger (312) laterally outwardly of the corresponding receptacle (240) of guide tack (200), and may relax the operator's grip of the proximal portions (308) of lever arms (302) to allow lever arms (302) to resiliently transition toward the closed state, such that each distal finger (312) may be received within the corresponding receptacle (240) of guide tack (200), as shown in FIG. 9A. The operator may then install guide tack (200) on the eye (101) by manipulating instrument (300). When the operator wishes to release guide tack (200) from instrument (300) (e.g., when legs (210) of guide tack (200) are fully inserted into the eye (101) and head (220) of guide tack (200) is abutting the surface (105) of the sclera (104)), the operator may again perform a pinching motion with the operator's thumb and index finger to transition lever arms (302) to the open state to thereby increase the gap between distal fingers (312) such that each distal finger (312) is retracted out of the corresponding receptacle (240) of guide tack (200), as shown in FIG. 9B.

### V. Example of Marking Instrument

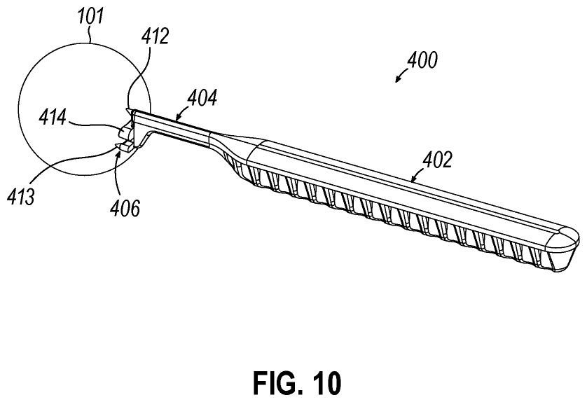

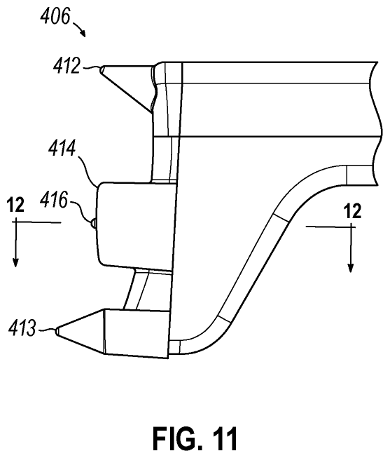

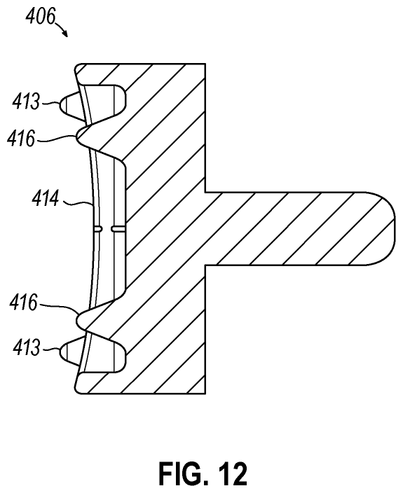

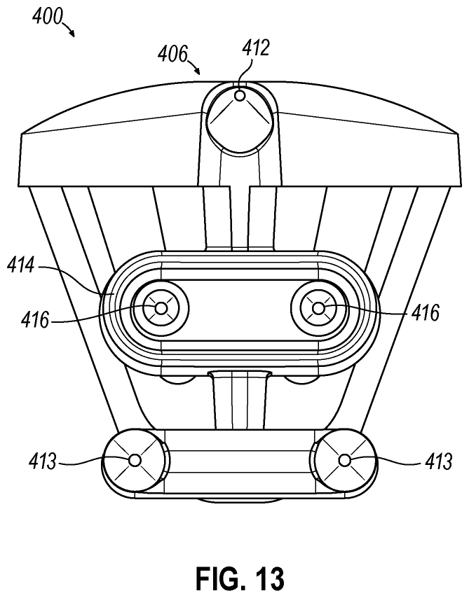

FIGS. 10-13 show an example of a marking instrument (400) that may be used to mark the site where guide tack (200) is to be installed in the eye (101) and/or mark the site where a sclerotomy (107) is to be formed. Instrument (400) of this example includes a handle (402), a shaft (404), and a marking head (406). Handle (402) is sized and configured to be easily grasped and manipulated by an operator's handle (e.g., using a pencil grip).

In the example shown, marking head (406) includes a first prong (412), a pair of second prongs (413), an oblong marking feature (414), and a pair of third prongs (416). While prongs (412, 413, 416) are pointed in the present example, prongs (412, 413, 416) are configured to be atraumatic such that prongs (412, 413, 416) will not pierce the sclera (104) when prongs (412, 413, 416) are urged against the surface (105) of the sclera (104) to mark the surface (105) as described herein. First prong (412) is configured to be positioned at the limbus of the eye (101) and thereby serve as a positional reference for the other prongs (413, 416) and marking feature (414) of marking head (410). Second prongs (413) are positioned and spaced to correspond with the location and length of the sclerotomy (107). Third prongs (416) are positioned and spaced to correspond with the location and spacing of the legs (210) of guide tack (200). Third prongs (416) are thus positioned to correspond with the location of the pars plana when first prong (412) is positioned on the limbus. Oblong marking feature (414) is sized and configured to correspond with the configuration of the head (220) of guide tack (200). Oblong marking feature (414) surrounds third prongs (416).

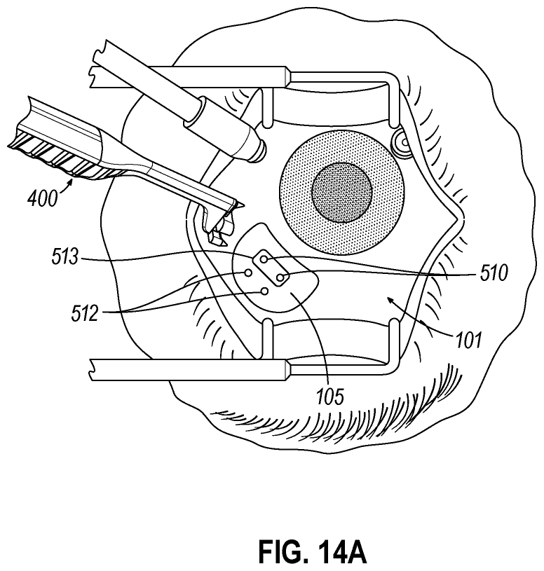

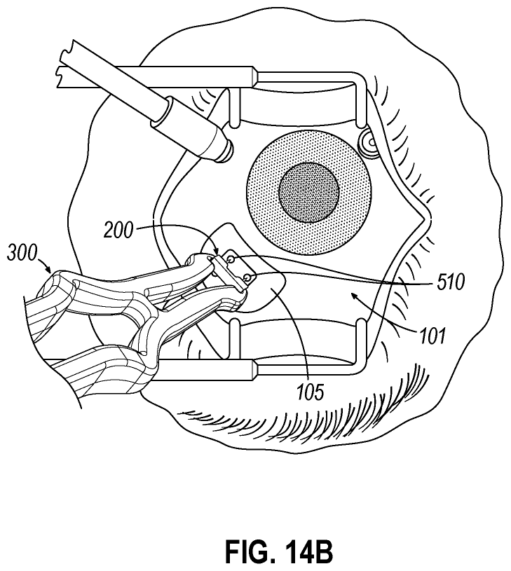

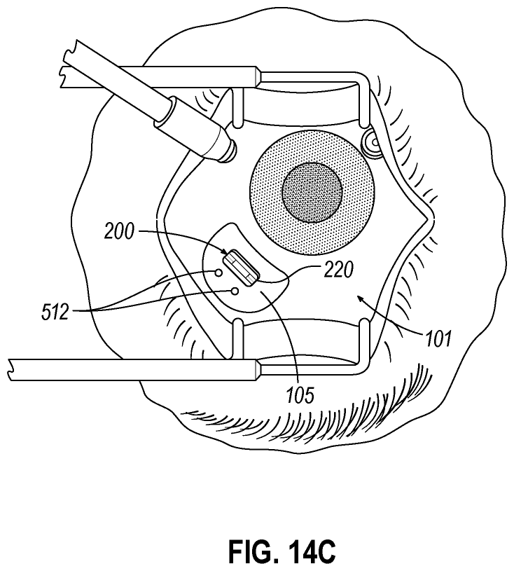

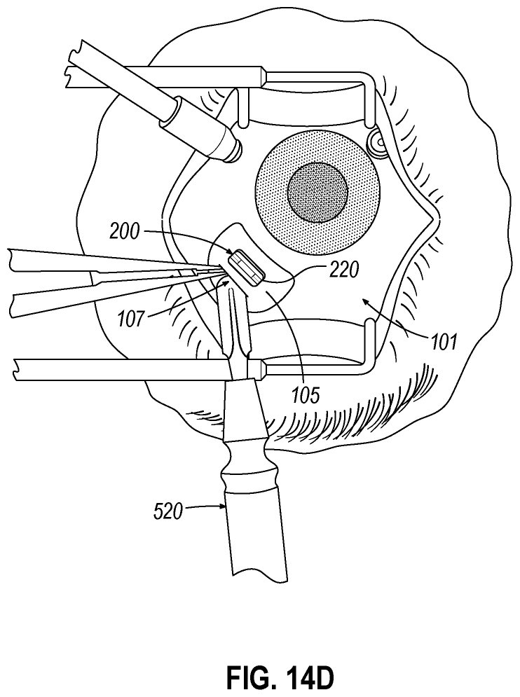

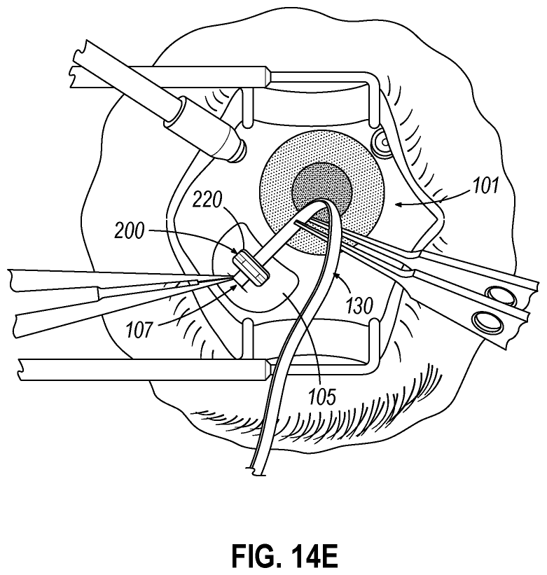

In use, the operator may press marking head (410) against an inkpad, then position first prong (412) at the limbus of the eye (101) and press marking head (410) against the surface (105) of the sclera (104) to leave markings in a manner similar to that described below with reference to FIG. 14C. The operator may then observe the positions of the markings left by third prongs (416) and insert the legs (210) of guide tack (200) at the locations of those markings. When guide tack (200) is fully seated against the surface (105) of the sclera (104), the operator may verify proper positioning by observing correspondence between the marking left by oblong marking feature (414) and the head (220) of guide tack (200). The operator may then form the sclerotomy (107) by using a scalpel to cut between the markings left by second prongs (413).

It will be appreciated that marking head (410) may have any other suitable configuration for leaving markings on the eye (101) in any other desired arrangement. For example, marking head (410) may be configured and operable in accordance with at least some of the teachings of U.S. Pat. No. 11,000,410, entitled “Guide Apparatus for Tangential Entry into Suprachoroidal Space,” issued May 11, 2021, the disclosure of which is incorporated by reference herein, in its entirety.

### VI. Example of Method of Using Guide Tack

FIGS. 14A-14E show an example of a procedure in which guide tack (200) is used to guide cannula (130) into a patient's eye (101). As shown in FIG. 14A, the sclera of the patient's eye (101) is accessed by dissecting the conjunctiva by incising a flap in the conjunctiva and pulling the flap posteriorly. After such a dissection is completed, the exposed surface (105) of the sclera (104) may optionally be blanched using a cautery tool to minimize bleeding. Once conjunctiva dissection is complete, the exposed surface (105) of the sclera (104) may optionally be dried using a WECK-CEL or other suitable absorbent device. Marking head (406) of marking instrument (400) is pressed against exposed surface (105) to apply first and second pairs of marks (510, 512) and an oblong mark (513) to exposed surface (105). By way of example only, a pigment material may be first applied to marking head (406) (e.g., by pressing marking head (406) against an ink pad), such that marking head (406) leaves some of the pigment on exposed surface (105) to provide marks (510, 512, 513). In the present example, marks (510) are located on the pars plana region of the eye (101).

As shown in FIG. 14B, the operator may then use deployment instrument (300) to install guide tack (200) in eye (101). In particular, the operator may align tips (212) with marks (510) and then press guide tack (200) toward eye (101), thereby piercing eye (101) with tips (212). The operator may further urge guide tack (200) toward eye (101) until the bottom surface (224) of head (220) abuts exposed surface (105). The abutment of underside (430) with exposed surface (105) will ensure consistent spacing between upper notch surface (232) and surface (105), such that the height of the guide opening defined by guide notch (230) and surface (105) should not vary from procedure to procedure.

As shown in FIG. 14C, when the operator removes deployment instrument (300), guide tack (200) is anchored to the eye (101). In the present example, legs (210) are configured to have a length that is less than the thickness of the sclera (210), such that legs (210) do not pierce the choroid (106) when guide tack (200) is fully seated against the eye (101). This is shown in FIGS. 15C-15G, which are described in greater detail below. As noted above, in some versions, sharp tips (212) and/or other portions of legs (210) may be textured to provide improved gripping of the sclera (210) after guide tack (200) has been installed as shown in FIG. 14C.

As shown in FIG. 14D, the operator then uses a conventional scalpel (520) to form a sclerotomy (107). The sclerotomy (107) is formed between marks (512), such that marks (512) are used to identify the ends of the sclerotomy (107). By way of example only, the sclerotomy (107) may be approximately 3 mm long, extending center-to-center between marks (512). The sclerotomy (107) extends through the full scleral thickness, removing all scleral fibers. The sclerotomy (107) is performed with particular care to avoid penetration of the choroid (106). Thus, the sclerotomy procedure provides access to the space between sclera (104) and choroid (106). Once the sclerotomy (107) is made in the eye (101), a blunt dissection may optionally be performed to locally separate sclera (104) from choroid (106). Such a dissection may be performed using a small blunt elongate instrument, as will be apparent to those skilled in the art in view of the teachings herein.

After the sclerotomy (107) is formed, and as shown in FIG. 14E, cannula (130) is passed through the guide opening defined by exposed surface (105) and guide notch (230) of guide tack (200) and then through the sclerotomy (107). In particular, cannula (130) is inserted into the space between the sclera (104) and the choroid (106). As described above, guide tack (200) may stabilize cannula (130). Additionally, guide tack (200) maintains cannula (130) in a generally tangential orientation relative to sclerotomy (107). Such tangential orientation may reduce trauma as cannula (130) is guided through sclerotomy (107) to stabilize cannula (130) and to prevent damage to surrounding tissue. As cannula (130) is inserted into sclerotomy (107) through guide tack (200), the operator may use forceps or other instruments to further guide cannula (130) along an atraumatic path. Of course, use of forceps or other instruments is merely optional, and may be omitted in some examples.

In some versions, cannula (130) may further include at least one depth marker (not shown) on the outer surface of cannula (130). The depth marker may be configured such that the operator may visually observe the position of the depth marker in relation to guide tack (200) or in relation to the sclerotomy (107) to determine when cannula (130) has been inserted to a predetermined insertion depth. By way of example only, the depth marker may be positioned to correspond with an initial cannula (130) insertion depth of approximately 5 mm in relation to the sclerotomy (107). The depth marker may be configured in accordance with at least some of the teachings of U.S. Pat. No. 11,000,410, entitled “Guide Apparatus for Tangential Entry into Suprachoroidal Space,” issued May 11, 2021, the disclosure of which is incorporated by reference herein, in its entirety.

As yet another merely illustrative variation, cannula (130) may include an outwardly protruding depth stop feature that abuts guide tack (200) to physically restrict the depth of insertion of cannula (130) in the eye (101). In some such versions, the depth stop feature is configured to enable selective positioning of the depth stop feature along the length of cannula (130), such that the operator may select a desired depth of insertion before passing cannula (130) through the guide opening. Again, one or more depth markers may be used to assist the operator in selecting a position for an adjustable depth stop feature along the length of cannula (130). Various suitable forms that a depth stop feature may take will be apparent to those skilled in the art in view of the teachings herein.

After reaching the state shown in FIG. 14E, and after cannula (130) has been inserted into the eye (101) to an appropriate depth, the procedure may continue as described below with reference to FIGS. 15D-15G. After the therapeutic agent (341) is injected into the subretinal space, cannula (130) may be withdrawn from the eye (101) and guide tack (200) may also be removed from the eye (101). The sclerotomy (107) may then be closed using any suitable conventional techniques. As noted below, the site where needle (150) penetrated through choroid (106) is self-sealing, such that no further steps need be taken to seal the delivery site through choroid (106). As also noted above, the sites where legs (210) penetrated through the eye (101) are also self-sealing, such that no further steps need be taken to seal the puncture sites where legs (210) were inserted.

### VII. Example of Procedure for Subretinal Administration of Therapeutic Agent

FIGS. 15A-15G show an example of a procedure that may be carried out using the above-described equipment, to deliver a therapeutic agent to the subretinal space of the eye (101) from a suprachoroidal approach. By way of example only, the method described herein may be employed to treat macular degeneration and/or other ocular conditions. Although the procedure described herein is discussed in the context of the treatment of age-related macular degeneration, no such limitation is intended or implied. For instance, in some alternative procedures, the same techniques described herein may be used to treat retinitis pigmentosa, diabetic retinopathy, and/or other ocular conditions. Additionally, the procedure described herein may be used to treat either dry or wet age-related macular degeneration, among other conditions.

In the present example, the procedure begins by an operator immobilizing tissue surrounding a patient's eye (101) (e.g., the eyelids) using an instrument such as speculum (162), and/or any other instrument suitable for immobilization. While immobilization described herein with reference to tissue surrounding eye (101), eye (101) itself may remain free to move. Once the tissue surrounding eye (101) has been immobilized, an eye chandelier port (514) is inserted into eye (101), as shown in FIG. 15A, to provide intraocular illumination when the interior of eye (101) is viewed through the pupil. In the present example, eye chandelier port (514) is positioned in the inferior medial quadrant such that a superior temporal quadrant sclerotomy may be performed. Eye chandelier port (514) is positioned to direct light onto the interior of eye (101) to illuminate at least a portion of the retina (108) (e.g., including at least a portion of the macula). As will be understood, such illumination corresponds to an area of eye (101) that is being targeted for delivery of therapeutic agent.

In the present example, only chandelier port (514) is inserted at the stage shown in FIG. 15A, without yet inserting an optical fiber (515) into port (514). In some other versions, an optical fiber (515) may be inserted into chandelier port (514) at this stage. In either case, a microscope may optionally be utilized to visually inspect the eye to confirm proper positioning of eye chandelier port (514) relative to the target site. Although FIG. 15A shows a certain positioning of eye chandelier port (514), eye chandelier port (514) may have any other suitable positioning as will be apparent to those skilled in the art in view of the teachings herein.

Once eye chandelier port (514) has been positioned, the sclera (104) may be accessed by dissecting the conjunctiva by incising a flap in the conjunctiva and pulling the flap posteriorly. After such a dissection is completed, the exposed surface of the sclera (104) may optionally be blanched using a cautery tool to minimize bleeding. Once conjunctiva dissection is complete, the exposed surface of the sclera (104) may optionally be dried using a WECK-CEL or other suitable absorbent device.

Marking instrument (400) may then be used to mark the eye (101), as described above. The operator may then use the marks (510, 512) created using the marking instrument (400) to deploy guide tack (200) and to perform a sclerotomy procedure, as shown in FIG. 15B, using a conventional scalpel (520) or other suitable cutting instrument.

The sclerotomy procedure forms a small incision (107) through sclera (104) of eye (101). The sclerotomy procedure is performed with particular care to avoid penetration of the choroid (106). Thus, the sclerotomy procedure provides access to the space between sclera (104) and choroid (106). Once the incision (107) is made in eye (101), a blunt dissection may optionally be performed to locally separate sclera (104) from choroid (106). Such a dissection may be performed using a small blunt elongate instrument, as will be apparent to those skilled in the art in view of the teachings herein.

With the sclerotomy procedure performed, an operator may insert cannula (130) of instrument (100) through the incision (107) and into the space between sclera (104) and choroid (106). As can be seen in FIG. 15C, cannula (130) is directed through guide tack (200) and into the incision. Guide tack (200) may stabilize cannula (130) during insertion. Additionally, guide tack (200) maintains cannula (130) in a generally tangential orientation relative to the sclera (104) and choroid (106) curvatures. Such tangential orientation may reduce trauma as cannula (130) is guided through the incision. As cannula (130) is inserted into the incision through guide tack (200), an operator may use forceps or other instruments to further guide cannula (130) along an atraumatic path. Of course, use of forceps or other instruments is merely optional, and may be omitted in some examples. Cannula (130) is advanced until distal end (132) is positioned near the targeted region of the subretinal space that is on the opposite side of the choroid (106). In the example shown, this targeted region is in the posterior portion of the eye (101); and cannula (130) conforms to the curvature of the eye (101) as cannula (130) advances from the anterior position to the posterior position. Various suitable ways of visualizing distal end (132) to thereby observe proper positioning of distal end (132) will be apparent to those skilled in the art in view of the teachings herein.

Although not shown, in some examples, cannula (130) may include one or more markers on the surface of cannula (130) to indicate various depths of insertion. While merely optional, such markers may be desirable to aid an operator in identifying the proper depth of insertion as cannula (130) is guided along an atraumatic path. For instance, the operator may visually observe the position of such markers in relation to guide tack (200) and/or in relation to the incision in the sclera (104) as an indication of the depth to which cannula (130) is inserted in eye (101). By way of example only, one such marker may correspond to an approximately 6 mm depth of insertion of cannula (130).

As shown in FIG. 15D, once cannula (130) is at least partially inserted into eye (101), an operator may insert an optical fiber (515) into eye chandelier port (514) if the fiber (515) had not yet been inserted at this stage. With eye chandelier port (514) in place and assembled with optical fiber (515), an operator may activate eye chandelier port (514) by directing light through optical fiber (515) to provide illumination of eye (101) and thereby visualize the interior of eye (101). Further adjustments to the positioning of cannula (130) may optionally be made at this point to ensure proper positioning relative to the area of geographic atrophy of retina (108). In some instances, the operator may wish to rotate the eye (101) to direct the pupil of the eye (101) toward the operator in order to optimize visualization of the interior of the eye (101) via the pupil.

FIGS. 15C-15D show cannula (130) as it is guided between sclera (104) and choroid (106) to position distal end (132) of cannula (130) at the delivery site for the therapeutic agent. In the present example, the delivery site corresponds to a generally posterior region of eye (101) adjacent to an area of geographic atrophy of retina (108). In particular, the delivery site of the present example is superior to the macula, in the potential space between the neurosensory retina and the retinal pigment epithelium layer. By way of example only, the operator may rely on direct visualization through a microscope directed through the pupil of eye (101) as cannula (130) is being advanced through the range of motion shown in FIGS. 15C-15D, with illumination provided through fiber (515) and port (514). Cannula (130) may be at least partially visible through the retina (108) and choroid (106) of eye (101). Visual tracking may be enhanced in versions where an optical fiber is used to emit visible light through the distal end of cannula (130).

Once cannula (130) has been advanced to the delivery site as shown in FIG. 15D, an operator may advance needle (150) of instrument (100) as described above by actuating knob (120). As can be seen in FIG. 15E, needle (150) is advanced relative to cannula (130) such that needle (150) pierces through choroid (106) without penetrating retina (108). Immediately prior to penetrating choroid (106), needle (150) may appear under direct visualization as “tenting” the surface of choroid (106). In other words, needle (150) may deform choroid (106) by pushing upwardly on choroid (106), providing an appearance like a tent pole deforming the roof of a tent. Such a visual phenomenon may be used by an operator to identify whether choroid (106) is about to be pierced and the location of any eventual piercing. The particular amount of needle (150) advancement sufficient to initiate “tenting” and subsequent piercing of choroid (106) may be of any suitable amount as may be determined by a number of factors such as, but not limited to, general patient anatomy, local patient anatomy, operator preference, and/or other factors. As described above, an example of a range of needle (150) advancement may be between approximately 0.25 mm and approximately 10 mm; or more particularly between approximately 2 mm and approximately 6 mm.

In the present example, after the operator has confirmed that needle (150) has been properly advanced by visualizing the tenting effect described above, the operator infuses a balanced salt solution (BSS) or other similar solution as needle (150) is advanced relative to cannula (130). Such a BSS may form a leading bleb (540) ahead of needle (150) as needle (150) is advanced through choroid (106). Leading bleb (540) may be desirable for two reasons. First, as shown in FIG. 15F, leading bleb (540) may provide a further visual indicator to an operator to indicate when needle (150) is properly positioned at the delivery site. Second, leading bleb (540) may provide a barrier between needle (150) and retina (108) once needle (150) has penetrated choroid (106). Such a barrier may push the retinal wall outwardly, thereby minimizing the risk of retinal perforation as needle (150) is advanced to the delivery site. In some versions, a foot pedal is actuated in order to drive leading bleb (540) out from needle (150). Alternatively, other suitable features that may be used to drive leading bleb (540) out from needle (150) will be apparent to those skilled in the art in view of the teachings herein.

Once the operator visualizes leading bleb (540), the operator may cease infusion of BSS, leaving a pocket of fluid as can be seen in FIG. 15F. Next, a therapeutic agent (542) may be infused by actuating fluid delivery system (180) or some other fluid delivery device as described in various references cited herein. The delivered therapeutic agent (542) may be any suitable therapeutic agent configured to treat an ocular condition. Some merely illustrative examples of suitable therapeutic agents may include, but are not necessarily limited to, drugs having smaller or large molecules, therapeutic cell solutions, certain gene therapy solutions, tissue plasminogen activators, and/or any other suitable therapeutic agent as will be apparent to those skilled in the art in view of the teachings herein. By way of example only, the therapeutic agent (542) may be provided in accordance with at least some of the teachings of U.S. Pat. No. 7,413,734, entitled “Treatment of Retinitis Pigmentosa with Human Umbilical Cord Cells,” issued Aug. 19, 2008, the disclosure of which is incorporated by reference herein, in its entirety. In addition to, or as an alternative to, being used to deliver a therapeutic agent (542), instrument (100) and variations thereof may be used to provide drainage and/or perform other operations.

In the present example, the amount of therapeutic agent (542) that is ultimately delivered to the delivery site is approximately 50 μL, although any other suitable amount may be delivered. In some versions, a foot pedal is actuated in order to drive agent (542) out from needle (150). Alternatively, other suitable features that may be used to drive agent (542) out from needle (150) will be apparent to those skilled in the art in view of the teachings herein. Delivery of therapeutic agent (542) may be visualized by an expansion of the pocket of fluid as can be seen in FIG. 15G. As shown, therapeutic agent (542) essentially mixes with the fluid of leading bleb (540) as therapeutic agent (542) is injected into the subretinal space.

Once delivery is complete, needle (150) may be retracted by rotating knob (120) in a direction opposite to that used to advance needle (150); and cannula (130) may then be withdrawn from eye (101). Because of the size of needle (150), the site where needle (150) penetrated through choroid (106) is self-sealing, such that no further steps need be taken to seal the delivery site through choroid (106). Guide tack (200) and chandelier (514) may be removed, and the incision in the sclera (104) may be closed using any suitable conventional techniques.

As noted above, the foregoing procedure may be carried out to treat a patient having macular degeneration. In some such instances, the therapeutic agent (542) that is delivered by needle (150) may comprise cells that are derived from postpartum umbilicus and placenta. As noted above, and by way of example only, the therapeutic agent (542) may be provided in accordance with at least some of the teachings of U.S. Pat. No. 7,413,734, the disclosure of which is incorporated by reference herein, in its entirety. Alternatively, needle (150) may be used to deliver any other suitable substance or substances, in addition to or in lieu of those described in U.S. Pat. No. 7,413,734 and/or elsewhere herein. By way of example only, therapeutic agent (542) may comprise various kinds of drugs including but not limited to small molecules, large molecules, cells, and/or gene therapies. It should also be understood that macular degeneration is just one merely illustrative example of a condition that may be treated through the procedure described herein. Other biological conditions that may be addressed using the instruments and procedures described herein will be apparent to those of ordinary skill in the art.

The procedure described above may be carried out in accordance with any of the teachings of U.S. Pat. No. 10,226,379, the disclosure of which is incorporated by reference herein, in its entirety; U.S. Pat. No. 9,949,874, entitled “Therapeutic Agent Delivery Device with Convergent Lumen,” issued Apr. 24, 2018, the disclosure of which is incorporated by reference herein, in its entirety; U.S. Pat. No. 9,925,088, entitled “Sub-Retinal Tangential Needle Catheter Guide and Introducer,” issued Mar. 27, 2018, the disclosure of which is incorporated by reference herein, in its entirety; U.S. Pat. No. 10,322,028, entitled “Method and Apparatus for Sensing Position Between Layers of an Eye,” issued Jun. 18, 2019, the disclosure of which is incorporated by reference herein, in its entirety; U.S. Pat. No. 10,064,752, entitled “Motorized Suprachoroidal Injection of Therapeutic Agent,” issued Sep. 4, 2018, the disclosure of which is incorporated by reference herein, in its entirety; U.S. Pat. No. 10,219,936, entitled “Therapeutic Agent Delivery Device with Advanceable Cannula and Needle,” issued Mar. 5, 2019, the disclosure of which is incorporated by reference herein, in its entirety; U.S. Pat. No. 10,258,502, entitled “Therapeutic Agent Delivery Device,” issued Apr. 16, 2019, the disclosure of which is incorporated by reference herein, in its entirety; and/or International Pub. No. WO 2022/136913, entitled “Ocular Cannula Guide,” published Jun. 30, 2022, the disclosure of which is incorporated by reference herein, in its entirety.

### VIII. Examples of Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An apparatus, comprising: (a) a pair of rigid legs extending parallel with each other along a plane, wherein each leg of the pair of legs has a sharp tip; and (b) a head, wherein the head includes: (i) a top surface, (ii) a bottom surface, (iii) a pair of end surfaces, (iv) a pair of side surfaces, (v) a guide notch extending upwardly from the bottom surface, wherein the guide notch is configured to cooperate with a surface of a sclera of a patient's eye to define a guide opening that is oriented transversely relative to the plane associated with the legs and that is sized to receive a cannula having a generally flat profile, and (vi) a pair of receptacles extending laterally inwardly from respective side surfaces of the pair of side surfaces, wherein each receptacle of the pair of receptacles is configured to engage a deployment instrument.

### Example 2

The apparatus of Example 1, wherein each leg of the pair of legs is textured to grip the sclera of the patient's eye.

### Example 3

The apparatus of any of Examples 1 through 2, wherein each receptacle of the pair of receptacles is at least partially defined by a corresponding laterally inner surface.

### Example 4

The apparatus of Example 3, wherein the laterally inner surface of each receptacle of the pair of receptacles is substantially vertical.

### Example 5

The apparatus of any of Examples 1 through 4, wherein each receptacle of the pair of receptacles is at least partially defined by a corresponding upper surface.

### Example 6

The apparatus of Example 5, wherein the upper surface of each receptacle of the pair of receptacles is tapered downwardly in a laterally inward direction.

### Example 7

The apparatus of any of Examples 1 through 6, wherein each receptacle of the pair of receptacles is at least partially defined by a corresponding pair of end surfaces.

### Example 8

The apparatus of Example 7, wherein the pair of end surfaces of each receptacle of the pair of receptacles are tapered toward each other in a laterally inward direction.

### Example 9

The apparatus of any of Examples 1 through 8, wherein each receptacle of the pair of receptacles is open along a bottom thereof.

### Example 10

The apparatus of any of Examples 1 through 9, wherein each side surface of the pair of side surfaces is U-shaped.

### Example 11

The apparatus of any of Examples 1 through 10, wherein the guide notch is defined by an upper notch surface and a pair of outer notch surfaces.

### Example 12

The apparatus of Example 11, wherein a mid-region of the upper notch surface is spaced apart from the bottom surface of the head to define a height of the guide opening.

### Example 13

The apparatus of Example 12, wherein the height is between about 0.27 mm and about 0.43 mm.

### Example 14

The apparatus of Example 13, wherein the height is about 0.35 mm.

### Example 15

A system comprising: (a) the apparatus of any of Examples 1 through 14; and (b) a deployment instrument, wherein the deployment instrument comprises a pair of distal fingers, wherein each distal finger of the pair of distal fingers is configured to be received within a corresponding receptacle of the pair of receptacles.

### Example 16

The system of Example 15, wherein the deployment instrument further comprises a pair of lever arms pivotably coupled to each other by a hinge such that the pair of lever arms are configured to pivot relative to each other about the hinge between a closed state and an open state, wherein each distal finger of the pair of distal fingers is positioned distal of the hinge.

### Example 17

The system of Example 16, wherein the pair of distal fingers are configured to move away from each other when the pair of lever arms are pivoted relative to each other about the hinge from the closed state to the open state.

### Example 18

The system of any of Examples 16 through 17, wherein each lever arm of the pair of lever arms includes a finger rest positioned proximal of the hinge, wherein the finger rests are operable to move toward each other to pivot the pair of lever arms relative to each other about the hinge from the closed state to the open state.

### Example 19

The system of any of Examples 16 through 18, further comprising a marking instrument, wherein the marking instrument comprises: (i) a handle, (ii) a shaft, and (iii) a marking head configured to mark a sclerotomy site on the patient's eye.

### Example 20

A system comprising: (a) the apparatus of any of Examples 1 through 14; and (b) a marking instrument, wherein the marking instrument comprises: (i) a handle, (ii) a shaft, and (iii) a marking head configured to mark a sclerotomy site on the patient's eye.

### Example 21

The system of Example 20, wherein the marking head includes a first prong configured to be positioned at a limbus of the patient's eye.

### Example 22

The system of any of Examples 20 through 21, wherein the marking head includes a pair of second prongs positioned and spaced to mark the sclerotomy site.

### Example 23

The system of any of Examples 20 through 22, wherein the marking head includes an oblong marking feature sized and configured to correspond with a profile of the head of the apparatus.

### Example 24

The system of any of Examples 20 through 23, wherein the marking head includes a pair of third prongs positioned and spaced to mark insertion sites for the pair of legs of the apparatus.

### Example 25

An apparatus, comprising: (a) a proximal end; (b) a pair of lever arms extending distally from the proximal end and including respective distal fingers; (c) a hinge pivotably coupling the pair of lever arms to each other, such that each lever arm of the pair of lever arms includes a proximal portion that is proximal of the hinge and a distal portion that is distal of the hinge, wherein the pair of lever arms are pivotable relative to each other about the hinge, via approximation of the proximal portions toward each other, from a closed state in which the distal fingers are spaced apart from each other by a gap sized to accommodate a portion of a guide tack that is grasped by the distal fingers, and an open state in which the gap between the distal fingers is increased to release the guide tack from the distal fingers.

### Example 26

The apparatus of Example 25, wherein each distal finger of the pair of distal fingers is positioned distal of the hinge.

### Example 27

The apparatus of any of Examples 25 through 26, wherein the hinge includes a living hinge.

### Example 28

The apparatus of any of Examples 25 through 27, wherein each lever arm of the pair of lever arms includes a finger rest positioned proximal of the hinge, wherein the finger rests are operable to move toward each other to pivot the pair of lever arms relative to each other about the hinge from the closed state to the open state.

### Example 29

A system, comprising: (a) the apparatus of any of Examples 25 through 28; and (b) the guide tack, wherein the guide tack is configured to guide a cannula relative to a patient's eye, wherein the guide tack is grasped by the distal fingers of the apparatus.

### Example 30

A method of inserting a cannula into an eye of a patient, the method comprising: (a) inserting a pair of distal fingers of a deployment instrument into corresponding receptacles of a guide tack to thereby grasp the guide tack with the deployment instrument; (b) inserting legs of the guide tack into a pars plana region of the eye, wherein the guide tack further includes a head secured to upper ends of the legs, wherein the head includes a guide notch that cooperates with a surface of the eye to define a guide opening oriented transversely relative to a plane defined between the legs; (c) forming a sclerotomy near the guide tack; (d) inserting a cannula through the guide opening; and (e) inserting the cannula through the sclerotomy, wherein the guide tack is configured to guide the cannula through the sclerotomy at a substantially tangential orientation.

### IX. Miscellaneous

To the extent that several examples herein are described in the context of a cannula guide being positioned near an already-formed scleral incision, it should be understood that other kinds of procedures may be employed. For instance, in some variations of the procedures described herein, the cannula guide may be secured to the eye (20) first; and then the scleral incision may be formed after the cannula guide has been secured to the eye (20). Other suitable steps and sequences that may be carried out in procedures that include a combination of a scleral incision and a cannula guide will be apparent to those skilled in the art in view of the teachings herein.

It should be understood that any of the versions of the instruments described herein may include various other features in addition to or in lieu of those described above. By way of example only, any of the devices herein may also include one or more of the various features disclosed in any of the various references that are incorporated by reference herein.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by an operator immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one skilled in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1. An apparatus, comprising:
(a) a pair of rigid legs extending parallel with each other along a plane, wherein each leg of the pair of legs has a sharp tip; and
(b) a head, wherein the head includes:
(i) a top surface,
(ii) a bottom surface,
(iii) a pair of end surfaces,
(iv) a pair of side surfaces,
(v) a guide notch extending upwardly from the bottom surface, wherein the guide notch is configured to cooperate with a surface of a sclera of a patient's eye to define a guide opening that is oriented transversely relative to the plane associated with the legs and that is sized to receive a cannula having a generally flat profile, and
(vi) a pair of receptacles extending laterally inwardly from respective side surfaces of the pair of side surfaces, wherein each receptacle of the pair of receptacles is configured to engage a deployment instrument.

2. The apparatus of claim 1, wherein each leg of the pair of legs is textured to grip the sclera of the patient's eye.

3. The apparatus of claim 1, wherein each receptacle of the pair of receptacles is at least partially defined by a corresponding laterally inner surface.

4. The apparatus of claim 3, wherein the laterally inner surface of each receptacle of the pair of receptacles is substantially vertical.

5. The apparatus of claim 1, wherein each receptacle of the pair of receptacles is at least partially defined by a corresponding upper surface.

6. The apparatus of claim 5, wherein the upper surface of each receptacle of the pair of receptacles is tapered downwardly in a laterally inward direction.

7. The apparatus of claim 1, wherein each receptacle of the pair of receptacles is at least partially defined by a corresponding pair of end surfaces.

8. The apparatus of claim 7, wherein the pair of end surfaces of each receptacle of the pair of receptacles are tapered toward each other in a laterally inward direction.

9. The apparatus of claim 1, wherein each receptacle of the pair of receptacles is open along a bottom thereof.

10. The apparatus of claim 1, wherein each side surface of the pair of side surfaces is U-shaped.

11. The apparatus of claim 1, wherein the guide notch is defined by an upper notch surface and a pair of outer notch surfaces.

12. The apparatus of claim 11, wherein a mid-region of the upper notch surface is spaced apart from the bottom surface of the head to define a height of the guide opening.

13. The apparatus of claim 12, wherein the height is between about 0.27 mm and about 0.43 mm.

14. The apparatus of claim 13, wherein the height is about 0.35 mm.

15. A system comprising:
(a) the apparatus of claim 1; and
(b) a deployment instrument, wherein the deployment instrument comprises a pair of distal fingers, wherein each distal finger of the pair of distal fingers is configured to be received within a corresponding receptacle of the pair of receptacles.

16. The system of claim 15, wherein the deployment instrument further comprises a pair of lever arms pivotably coupled to each other by a hinge such that the pair of lever arms are configured to pivot relative to each other about the hinge between a closed state and an open state, wherein each distal finger of the pair of distal fingers is positioned distal of the hinge, wherein the pair of distal fingers are configured to move away from each other when the pair of lever arms are pivoted relative to each other about the hinge from the closed state to the open state, wherein each lever arm of the pair of lever arms includes a finger rest positioned proximal of the hinge, wherein the finger rests are operable to move toward each other to pivot the pair of lever arms relative to each other about the hinge from the closed state to the open state.

17-19. (canceled)

20. A system comprising:
(a) the apparatus of any claim 1; and
(b) a marking instrument, wherein the marking instrument comprises:
(i) a handle,
(ii) a shaft, and
(iii) a marking head configured to mark a sclerotomy site on the patient's eye.

21. The system of claim 20, wherein the marking head includes:
(A) a first prong configured to be positioned at a limbus of the patient's eye,
(B) a pair of second prongs positioned and spaced to mark the sclerotomy site,
(C) an oblong marking feature sized and configured to correspond with a profile of the head of the apparatus, and
(D) a pair of third prongs positioned and spaced to mark insertion sites for the pair of legs of the apparatus.

22-24. (canceled)

25. An apparatus, comprising:
(a) a proximal end;
(b) a pair of lever arms extending distally from the proximal end and including respective distal fingers;
(c) a hinge pivotably coupling the pair of lever arms to each other, such that each lever arm of the pair of lever arms includes a proximal portion that is proximal of the hinge and a distal portion that is distal of the hinge,
wherein the pair of lever arms are pivotable relative to each other about the hinge, via approximation of the proximal portions toward each other, from a closed state in which the distal fingers are spaced apart from each other by a gap sized to accommodate a portion of a guide tack that is grasped by the distal fingers, and an open state in which the gap between the distal fingers is increased to release the guide tack from the distal fingers.

26-29. (canceled)

30. A method of inserting a cannula into an eye of a patient, the method comprising:
(a) inserting a pair of distal fingers of a deployment instrument into corresponding receptacles of a guide tack to thereby grasp the guide tack with the deployment instrument;
(b) inserting legs of the guide tack into a pars plana region of the eye, wherein the guide tack further includes a head secured to upper ends of the legs, wherein the head includes a guide notch that cooperates with a surface of the eye to define a guide opening oriented transversely relative to a plane defined between the legs;
(c) forming a sclerotomy near the guide tack;
(d) inserting a cannula through the guide opening; and
(e) inserting the cannula through the sclerotomy, wherein the guide tack is configured to guide the cannula through the sclerotomy at a substantially tangential orientation.
